(19) United States
(12) Patent Application Publication
(10) Pub. No.: US 2008/0234614 A1
(43) Pub. Date: Sep. 25, 2008

(54) Injury immobilization device

(76) Inventor: Amit V. Patel, Yorktown Heights, NY (US)

Correspondence Address: AMIT V. PATEL, 2289 Willowway Street, Yorktown Heights, NY, 10598, US

(21) Appl. No.: 12/154,789

(22) Filed: May 27, 2008

Related U.S. Application Data
(62) Division of application No. 10/706,638, filed on Nov. 12, 2003.

(51) Int. Cl. A61F 5/00

(52) U.S. Cl. 602/5

(57) ABSTRACT

A device is provided that is capable of being mounted on both sides of an injured joint to immobilize the joint, typically as a means of providing emergency care to a patient. The inventive device has plates that are pivotally attached to each other so that the device can be placed against the patient's body and secured to body parts that are on opposing sides of the injured joint, without substantially moving the injured joint. In this manner, additional trauma, pain and/or injury to the patient is avoided or minimized while emergency care is being provided. According to one aspect of the invention, the apparatus has a first plate adapted for engaging a limb of the body, and a second plate adapted for engaging a body part to which the limb is connected via the injured joint. The first plate has a first end portion, and the second plate has a second end portion. The apparatus also has a lockable joint connecting the first end portion of the first plate and the second end portion of the second plate.

### RELATED APPLICATION

This application is a divisional of U.S. patent application Ser. No. 10/706,638 filed on Nov. 12, 2003.

### TECHNICAL FIELD

The present invention relates generally to pre-hospital injury or trauma care devices, and in particular, the invention provides an apparatus for immobilizing an injured body part.

### DESCRIPTION OF RELATED ART

When a person is injured, a key element of pre-hospital treatment is immobilization of the injury, i.e., the injury is secured so that the patient cannot move the injured body part. If an injury is effectively immobilized, and therefore completely inhibits motion in any direction, the patient is protected from further unnecessary discomfort and further injury.

For example, when a person receives a shoulder injury, various types of injury may be present. There may be tissue or muscular damage, a dislocation, or a fracture. In any of these scenarios, EMS (Emergency Medical Services) care providers want to immobilize the patient prior to transporting the patient to a hospital. There are many muscles surrounding the shoulder area, which can be pulled or torn. Shoulder dislocations are very common. When a shoulder fracture occurs, there are many ways in which the injury may manifest itself. The proximal (closest to the joint) end of the humerus, the scapula, or the clavicle may be any or all of the effected bones. EMS care providers can only guess what the injury may be, and then effectively immobilize the entire shoulder.

Shoulder injuries are very common. These types of injuries are often caused by sport-related accidents or PIAA's (Personal Injury Auto Accidents). Sources of these injuries often incorporate a significant MOI (mechanism of injury). For example, a baseball player may be hit in the shoulder by a foul-ball, or an individual in a motor vehicle may hit his or her shoulder by crashing into a stationary or moving object. The MOI could be determined by the possible rate of travel of the baseball, or the damage to the vehicle and its rate of travel. Shoulder injuries are very common, and can be simple, or very complex.

When treating a patient, EMS care providers are trained to not move their patients prior to careful physical assessment and treatment of any and all injuries found. Not moving the patient may be crucial, if a significant MOI is suspected or present. If an individual crashed his or her car into a streetlight, for example, aside from the shoulder pain he or she may be complaining of, the patient may have suffered from a neck injury, or a spinal injury. Moving a patient who has an injury of this type could leave him or her paralyzed or could be fatal. Another reason why patients are not immediately moved from where they are found is that EMS care providers cannot see under the skin. They cannot identify what type of injury they are dealing with. For instance; a shoulder fracture could be anything varying from a crack to a splinter fracture, in which the bone splinters like a toothpick. If a patient is found on the ground, with his or her hand extended outward, moving the patient's arm toward the anterior portion of the body could cause splintered bone fragments to shift, causing increased pain and trauma and potentially causing extensive muscular tissue and even bone damage.

When dealing with shoulder injuries today, EMS care providers typically treat their patients by securing injured shoulders using cravats and slings, or by using special air mattress-like devices. An example of an air mattress-like device is distributed by Medical Devices International and is called the “EMS IMMOBILE-VAC™ Pediatric/Universal Mattress.”

Currently, there is no known standard device that is universally used for shoulder injuries. In order to immobilize a patient's shoulder, EMS care providers must move the injured extremity towards the patient's body, where they can use a sling and multiple cravats to tie down the arm. In this process, there are two problems. The patient must be moved from the original position in which he or she was found; and using a sling with cravats does not completely immobilize the shoulder. Medical professionals can benefit from the use of a device which can be used as a standard for these types of injures and at the same time can be used in accordance with basic injury management protocols. There is also a need for an immobilization device that fits the contours of the patient, allowing other EMS devices to be additionally used. Furthermore, there exists a need for a device that can be used as a universal tool for a variety of different types of joint injuries in pre-hospital care situations. In addition, there is a need for an immobilization device that is small and easily portable.

### SUMMARY OF THE INVENTION

The invention provides a device that is capable of being mounted on both sides of an injured joint to immobilize the joint, typically as a means of providing emergency care to a patient. The inventive device has plates that are pivotally attached to each other so that the device can be placed against the patient's body and secured to body parts that are on opposing sides of the injured joint, without substantially moving the injured joint. In this manner, additional trauma, pain and/or injury to the patient is avoided or minimized while emergency care is being provided.

According to one aspect of the invention, an apparatus is provided for immobilizing an injured joint of a body. The apparatus has a first plate adapted for engaging a limb of the body, and a second plate adapted for engaging a body part to which the limb is connected via the injured joint. The first plate has a first end portion, and the second plate has a second end portion. The apparatus also has a lockable joint connecting the first end portion of the first plate and the second end portion of the second plate. At least one securing mechanism may be attached to one or both plates.

In another aspect of the invention, an apparatus for immobilizing an injured joint of a body comprises a first plate adapted for engaging a limb of the body, the first plate having a first end portion, and a second plate adapted for engaging a body part to which the limb is connected via the injured joint, the second plate having a second end portion. The second end portion is pivotably attached to the first end portion of the first plate. The apparatus also has a support mechanism extending between a back surface of the first plate and a back surface of the second plate, for maintaining the first plate in a fixed position relative to the second plate.

Another aspect of the invention provides apparatus for immobilizing an injured joint of a human body. The apparatus has a first plate adapted for engaging a limb of the body, the first plate having a first end portion; and a second plate adapted for engaging another body part to which the limb is connected via the injured joint, the second plate having a second end portion. The apparatus also has a hinge pivotably joining the first end portion of the first plate and the second end portion of the second plate. In addition, the apparatus includes means for securing the first plate to the limb of the body and means for securing the second plate to the other body part. An adjustable support mechanism has first and second opposite ends, such that the first end is attachable to a back surface of the first plate, and the second end is attachable to a back surface of the second plate.

The support mechanism may be a rod having an adjustable or fixed length. If the rod is adjustable, as in the case of a telescoping rod, the support mechanism may further include a lock to secure the rod at a desired length. The rod may be detachable from either the first plate or the second plate. According to an embodiment of the invention wherein one end of the rod is detachable, an adjustment mechanism may be provided on the back surface of one of the plates for receiving and holding the detachable end of the rod, wherein the adjustment mechanism facilitates locking the first plate and the second plate at a desired orientation with respect to each other.

These and other aspects of the invention will become apparent from the following detailed description of illustrative embodiments thereof, which is to be read in connection with the accompanying drawings.

### BRIEF DESCRIPTION OF THE DRAWINGS

FIG. 1 is a diagram illustrating an injury immobilization device in accordance with a preferred embodiment of the invention.

FIG. 2 is a diagram illustrating a second embodiment of the injury immobilization device.

FIG. 3 is a side-view diagram of FIG. 2.

FIG. 4 shows another embodiment of the invention, with a support.

FIG. 5 is a diagram of a close-up of the joint portrayed in FIGS. 1-4.

FIG. 6 illustrates an additional embodiment of the invention, in which straps are used.

### DETAILED DESCRIPTION

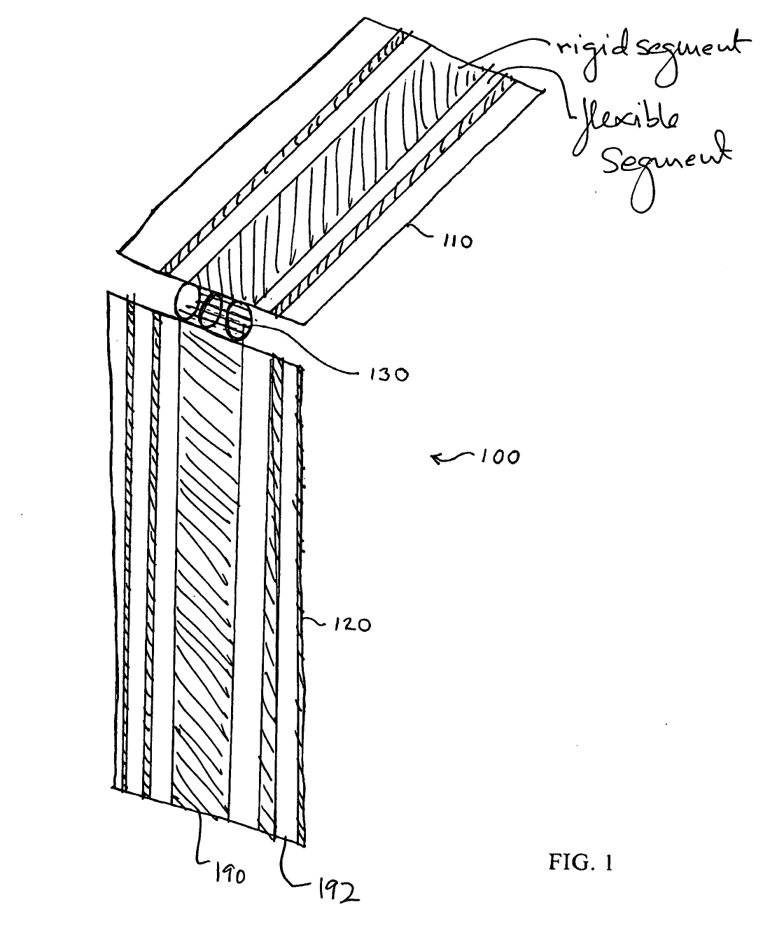

FIG. 1 illustrates a frontal view of an injury immobilization device 100 composed of two plates 110, 120 connected together by a pivotable joint 130. The invention may be used to immobilize various different joints of a body that have been injured, such as, for example, a shoulder, elbow, knee or hip. According to a preferred use of the invention, an emergency care provider may use the inventive device on an injured person who is suspected of having a bone fracture in or around the region of a body joint. If movement of the injured joint is desired to be kept to a minimum, the invention is capable of being oriented around the injured body joint and secured to the patient without substantially moving the joint. When used in this manner, the inventive device can prevent further injury and pain to the patient while the patient is being treated for other injuries and/or is being transported to a location where additional treatment can be provided.

Referring back to FIG. 1, both plates 110, 120 may be the same size or different sizes. According to one embodiment of the invention, the top plate 110 is approximately 10 inches wide and 10 inches long. To immobilize a shoulder injury, this plate 110 may be used to rest a patient's arm on. The upper-arm (humerus) on the tricep and elbow side may rest on this plate. Alternatively, for example, if a knee injury is to be immobilized, this plate 110 could support the lower-leg, or tibia and fibula, and be placed on the posterior side of the leg. In one embodiment of the invention, the bottom plate 120 is also approximately 10 inches wide and 10 inches wide. Those of ordinary skill in the art will recognize that the size and dimensions of the plates may vary according to the intended uses of the device. For example, biomedical and biomechanical data may be helpful in considering the particular materials and dimensions of an injury immobilization device so that the device is capable of supporting the weight of a human limb. Such types of data are widely available in publications such as, for instance, “Geometrical and Mass-inertial Characteristics of the Upper Human Limb,” published by the Centre of Biomedical Engineering, Bulgarian Academy of Science, as part of the Motco Data Project, and available on the Internet at http://www.motco.dir.bg/Data/MassInertial.html, the disclosure of which is incorporated by reference herein.

For shoulder injuries, plate 120 may rest on the side of the chest and pelvic regions. Plate 120 may help to support the top plate 110 and may be used in mounting the device onto the patient. For knee injuries, plate 120 may be attached to a patient's lower-leg or femur, on the posterior side of the leg. The joint by which the two plates are connected may be rigid or variable. The angle between the two plates may vary from as much as 180 degrees apart to as little as 0 degrees apart.

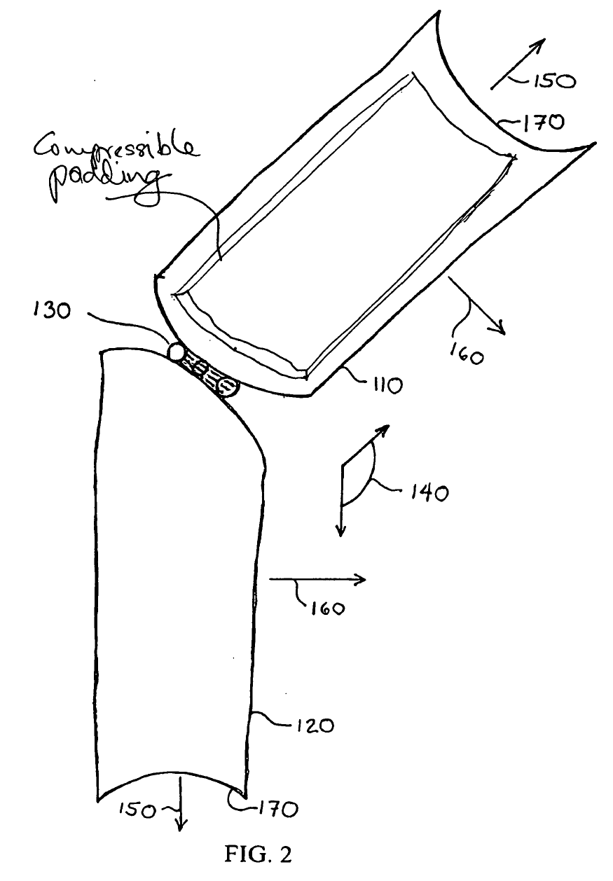

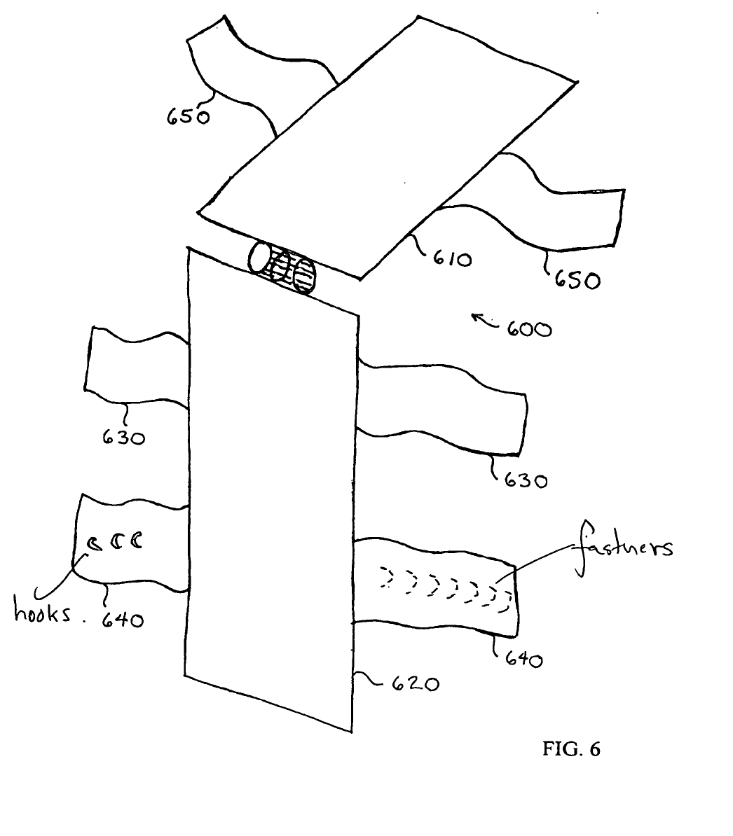

FIG. 2 illustrates a similar frontal view of the two plates 110, 120, and shows one possible position 140 of the plates with respect to each other. The plates 110, 120, which are flat when not in use as shown in FIG. 1, may mold to the contours of a patient's body in use as shown in FIG. 2. In FIG. 2 the plates 110, 120 are positioned so a shoulder may be immobilized at approximately 90 degrees. The joint 130 may be locked in place. The plates 110, 120 may fit the general contours of a patient's arm and lateral upper-body. Flexible and rigid properties of these plates may be based on a variety of different materials or combinations thereof, such as, for example, natural or synthetic fibers, fiberglass, carbon fiber, polycarbonate alloys, polymers, or moldable metals and foams. The plates 110, 120 may remain rigid in the lengthwise direction 150 while in the width-wise direction 160 the plates may be molded into a semi-cylindrical or concave shape 170 that is adapted to engage the curved portion of a body part such as an upper arm, side of a torso, thigh or leg. In the width-wise direction 160, plate 110 or 120 may be flexible or rigid. Metal or plastic strips (not shown) may also be provided as stiffening members secured in the lengthwise or crosswise direction (150 or 160, respectively) to maintain rigidity of the plates, particularly when the plates are made of a lightweight material that is not substantially rigid when used to support a heavy weight such as an arm or leg. Plate 110 and/or 120 may also be formed of multiple, rigid, parallel strips 190 that are flexibly connected together with a flexible material 192 such as a textile, plastic or other synthetic material, so that in the width-wise direction 160 plate 110 or 120 may be brought into close proximity to the curved portion of the body on which the device is to be secured. Alternatively, multiple, flexible plate segments 192 may be joined together with rigid connections 190 in order to form plates 110 and/or 120. Plate 110 or 120 may also be formed of a flexible material which includes one or more rigid supports in the lengthwise direction. If a plate is flexible, means are provided to ensure that the plate can be secured to the body part without falling off inadvertently, as described in further detail according to the description of FIG. 6 et seq.

One or both plates of the invention may be substantially rigid. The plate surface that comes into contact with the patient's body may be covered with a foam-like or compressible material that engages the patient's body part (e.g., leg or arm) and helps to hold the plate in place on the leg or arm part. Preferably the compressible material is covered with a fluid-resistant, hygienic coating or has a surface that can be cleaned or sterilized for re-use of the apparatus.

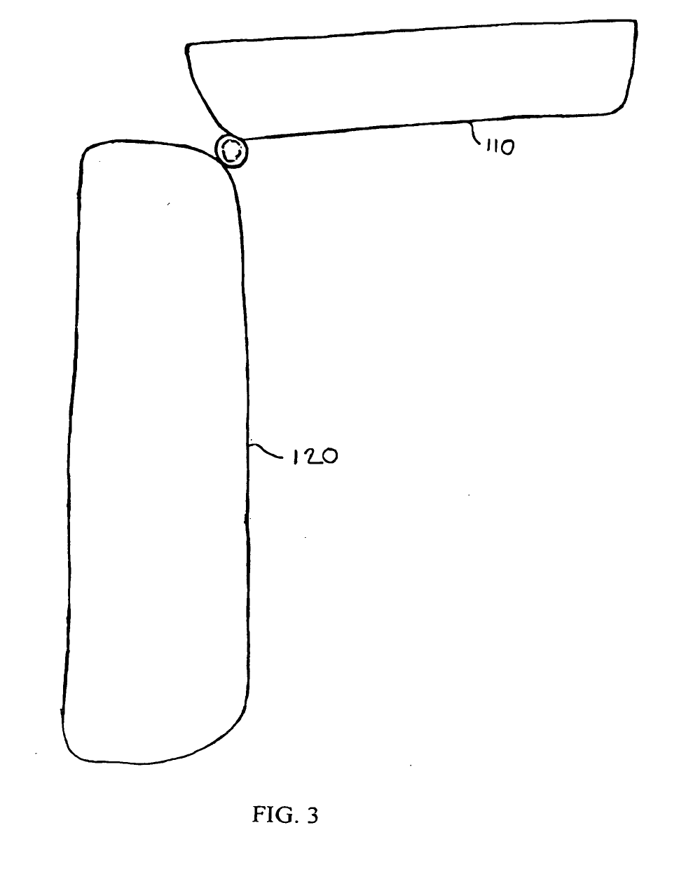

FIG. 3 illustrates a lateral view of FIG. 2. The two plates 110, 120 may be positioned as much as 180 degrees apart or as close together as about 0 degrees apart. The embodiment as illustrated in FIGS. 2 and 3 is preferred for immobilizing a shoulder injury. If the plates are oriented closer to 180 degrees apart, the device may be readily positioned for immobilizing a knee or hip injury.

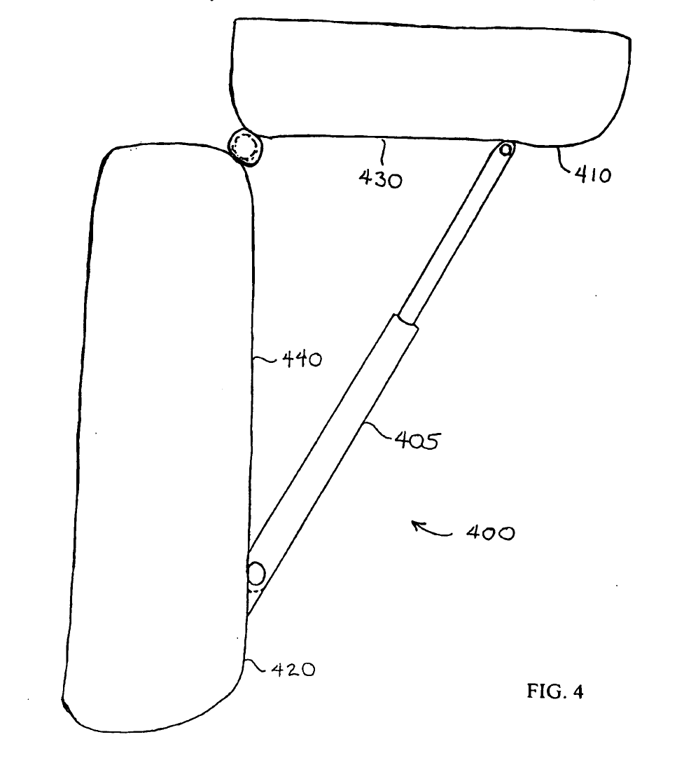

FIG. 4 illustrates an alternative embodiment 400 of the invention. According to this embodiment, a support 405 may be situated between the two plates 410 and 420, to add strength to the device. The support 405 may be a telescoping or sliding rod or pole, a rigid rod, or an expanding pole. This additional support 405 may change position or length and may be used to support the plates 410, 420 at any orientation or angle with respect to each other. This adjustable support 405 may be locked into place by a locking mechanism that is attached to or integral with the support. Alternatively, the locking mechanism may be attached to a back surface 430 or 440 of plate 410 or 420. According to one embodiment of the invention, the support 405 may be attachable or removable from one or both plates 410, 420 of the apparatus. Those of ordinary skill in the art will recognize that the location of the support may vary according to the specific size and/or shape of the device, as long as the support 405 is capable of preventing the plates from moving substantially from their proper orientation when they are used to immobilize an injured joint.

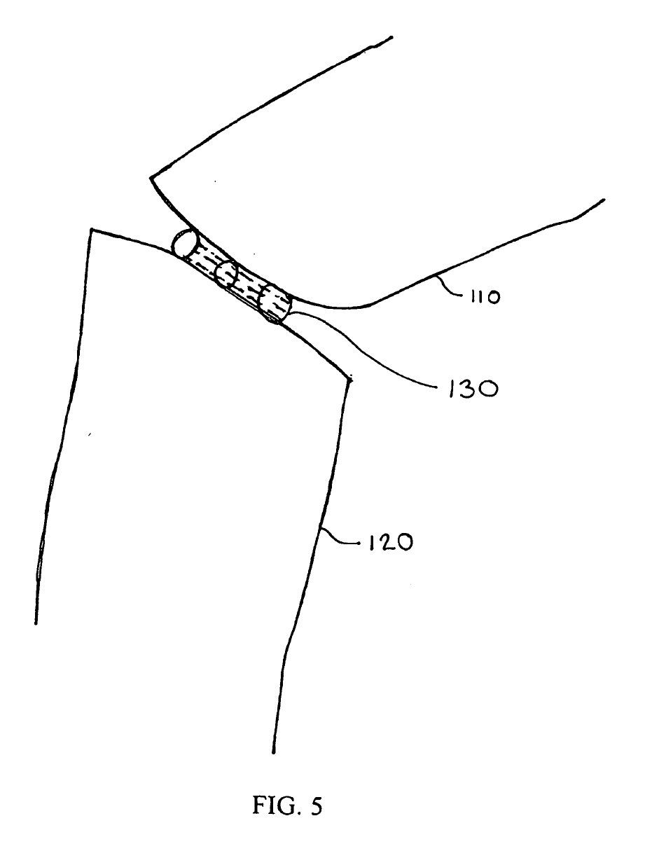

FIG. 5 illustrates an enlargement of joint 130. The joint connects the top and bottom plates 110, 120 together. This joint may be a free-moving joint, such as a hinge, or a flexible extension of at least one of the plates (110 or 120, 410 or 420), or it may be a lockable joint (not shown). Various components or combinations of components may be used to form a lockable joint, such as, for example, meshing teeth, or a removable pin or wedge or other mechanical part familiar to a person of ordinary skill in the art. The joint may be composed of either rigid material, such as metal, plastic or other natural or synthetic material; or the joint may be flexible material such as plastic, and it may be integral with the plates. According to a preferred embodiment of the invention, a portion of the joint is capable of being detachable. If the joint is detachable, the two plates can be separated. If the injury immobilization device 100 or 400 is capable of being disassembled, the device may be more efficient, portable, and adaptable for use in the field.

FIG. 6 illustrates yet another embodiment of the invention in which straps may be used to affix the device 600 to a patient. Straps or sets of straps 630, 640, 650 may be attached to one or both plates 610, 620. If the embodiment of FIG. 6 is used to immobilize an injured shoulder, for example, the patient's torso may be secured using one or more principal straps 630, 640. According to the illustration of FIG. 6, a first strap 630 may be secured around the chest of a patient, and a second strap 640 may be secured around the patient's pelvis. One or more additional straps or sets of straps 650 may be affixed to the top plate 610 and may be strapped around a patient's humerus or elbow for an embodiment of the invention that is used to secure an injured shoulder.

If straps are added to the device, they may be made of nylon, webbing or other flexible material, and the straps may be used to secure the device to the patient's body with knots or with the addition of fasteners (not shown) such as hook and loop fasteners, snaps, hook and eye fasteners, clips, clamps, ties, buckles or any other fastener type that a person of ordinary skill in the art would recognize as appropriate for securing the plates of the apparatus against a body in order to immobilize an injured joint. The straps can vary in size and location on the plates, depending on the particular size and shape of the device.

The description of the present invention has been presented for purposes of illustration and description, and is not intended to be exhaustive or limited to the invention in the form disclosed. Many modifications and variations will be apparent to those of ordinary skill in the art. For example, although the most preferred embodiment of the invention is an apparatus for immobilizing a shoulder joint, the invention is also adapted for use as an immobilization device for many types of injuries to other body joints, such as the knee, hip, elbow, ankle or wrist.

Those of ordinary skill in the art will also recognize that the embodiment was chosen and described in order to best explain the principles of the invention, the practical application, and to enable others of ordinary skill in the art to understand the invention for various embodiments with various modifications as are suited to the particular use contemplated.

1. An apparatus for immobilizing an injured joint of a body comprising:
a first plate adapted for engaging a limb of the body, the first plate having a first end portion;
a second plate adapted for engaging a body part to which the limb is connected via the injured joint, the second plate having a second end portion;
a hinge for pivotably connecting an end portion of the first plate and an end portion of the second plate; and
a rod having an adjustable length, a first rod end and an opposite rod end, wherein the first rod end extends from the first plate and the opposite rod end extends from the second plate, and wherein the rod maintains the first plate in a fixed position relative to the second plate.

2. The apparatus of claim 1, further comprising at least one limb securing mechanism attached to the first plate and a second limb securing mechanism attached to the second plate.

3. The apparatus of claim 2, wherein the at least one limb securing mechanism comprises at least one strap

4. The apparatus of claim 2, wherein the at least one limb securing mechanism comprises a strap and a hook and loop fastener.

5. The apparatus of claim 1, further comprising, a compressible padding attached to a concave surface of the first plate and a compressible padding attached to a concave surface of the second plate.

6. The apparatus of claim 1, further comprising, a locking mechanism that locks the rod in a fixed position.

7. The apparatus of claim 1, wherein at least one of the first plate and the second plate is contoured for receiving a body part when the apparatus is in use.

8. The apparatus of claim 1, wherein at least one of the first plate and the second plate flexes into a concave orientation when the apparatus is in use.

9. The apparatus of claim 1, wherein the first plate is disassembles from the second plate when the apparatus is not in use.

10. An apparatus for immobilizing an injured joint of a body, comprising: a first plate adapted for engaging a limb of the body, the first plate having a first end portion; a second plate adapted for engaging a body part to which the limb is connected via the injured joint, the second plate having a second end portion pivotably attached to the first end portion of the first plate; and a support mechanism extending between a back surface of the first plate and a back surface of the second plate, for maintaining the first plate in a fixed position relative to the second plate.

11. The apparatus of claim 10, wherein the support mechanism comprises a rod having an adjustable length.

12. The apparatus of claim 11, wherein the support mechanism further comprises a lock for maintaining the rod at a desired length.

13. The apparatus of claim 10, wherein the support mechanism comprises a rod that is detachable from at least one of the first plate and the second plate.

14. The apparatus of claim 10, further comprising an adjustment mechanism on the back surface of the first plate for receiving an end of the rod, wherein the adjustment mechanism facilities locking the first plate and the second plate at a desired orientation with respect to each other.

15. The apparatus of claim 10, further comprising a hinge pivotably joining the first end portion of the first plate and the second end portion of the second plate; means for securing the first plate to the limb of the body; means for securing the second plate to the other body part; and an adjustable support mechanism having first and second opposite ends, the first end attachable to a back surface of the first plate, and the second end attachable to a back surface of the second plate.

16. The apparatus of claim 10 wherein
the first plate is flat when the apparatus is not in use, the first plate having a first end portion;
the second plate is flat when the apparatus is not in use, the second plate having a second end portion;
a hinge for pivotably connecting the first end portion of the first plate and the second end portion of the second plate,
wherein, in use for immobilizing an injured joint of a body, the first and second plates remain rigid in their lengthwise direction while in their width-wise directions the plates are molded into a semi-cylindrical or concave shape that is adapted to engage the curved portion of a body part.

17. The apparatus of claim 24 wherein the plates are formed of alternating rigid and flexible plate segments that provide rigidity in the lengthwise directions of the plates and flexibility in the width-wise directions of the plates.
